(19) United States
(12) Patent Application Publication
(10) Pub. No.: US 2026/0334652 A1
(43) Pub. Date: Oct. 29, 2026

(54) COSMETIC CONTAINER HAVING PRESSURE CONTROL FUNCTION

(71) Applicant: Amorepacific Corporation, Seoul (KR)

(72) Inventors: Jinsoo JEONG, Yongin-si (KR); Kyoungsik Kong, Yongin-si (KR); Hyunjun Kwon, Yongin-si (KR); Hyungtaek OH, Yongin-si (KR); Kyuseok Lee, Yongin-si (KR); Sungsoo Hong, Yongin-si (KR)

(73) Assignee: Amorepacific Corporation, Seoul (KR)

(21) Appl. No.: 19/533,436

(22) Filed: Feb. 9, 2026

(30) Foreign Application Priority Data
Feb. 14, 2025 (KR) 10-2025-0019416

(51) Int. Cl. A45D 34/00; A61K 8/67; B65D 51/16

(52) U.S. Cl. CPC A45D 34/00; A61K 8/676; B65D 51/1644; B65D 51/1672; A45D 2034/002; A45D 2200/051; A61K 2800/87

(57) ABSTRACT

A cosmetic container has a pressure control function. Specifically, a cosmetic container has a pressure control function capable of maintaining a pressure lower than a set pressure in an upright state in which a container stands, a horizontal state in which the container is laid down, and an inverted state in which the container stands upside down. According to an embodiment, it is possible to provide a cosmetic container having a pressure control function including: a body forming an accommodating space configured to store a content capable of generating gas over time; and a selective blocking member configured to selectively allow the gas to pass to outside according to a magnitude of pressure of the gas generated in the accommodating space.

### CROSS-REFERENCE TO RELATED APPLICATIONS

This application is based on and claims priority of Korean Patent Application No 10-2025-0019416, filed on Feb. 14, 2025 with the Korean Intellectual Property Office, the entire contents of which are hereby incorporated by reference.

### TECHNICAL FIELD

The present disclosure relates to a cosmetic container having a pressure control function. Specifically, the present disclosure relates to a cosmetic container having a pressure control function capable of maintaining pressure lower than a set pressure in an upright state in which a container stands, a horizontal state in which the container is laid down, and an inverted state in which the container stands upside down.

### BACKGROUND

In recent years, as people's interest in beauty has increased, functional cosmetics containing various ingredients such as vitamins have been released. Such cosmetic ingredients may be oxidized inside a cosmetic container over time, thereby generating gas and increasing internal pressure of the cosmetic container.

For example, cosmetics containing vitamin C generate carbon dioxide by reacting with oxygen, high temperature, and ultraviolet rays inside the cosmetic container.

As such, as an amount of carbon dioxide generated inside the cosmetic container increases, the internal pressure of the cosmetic container increases, and when the internal pressure of the cosmetic container exceeds a predetermined pressure, a problem occurs in that the cosmetic container ruptures or is damaged. In addition, there is a risk that the cosmetics may be deteriorated as the cosmetic container ruptures or is damaged, or that users may be injured by the damaged cosmetic container.

In order to solve such problems, a valve for discharging carbon dioxide has been formed in a cap part of a container in the related art.

However, a valve formed in the conventional cap may discharge carbon dioxide to outside in an upright state in which the container stands, but in a horizontal state in which the container is laid down, or an inverted state in which the container is turned upside down, a content is disposed toward the valve side, thereby causing a problem in that the content is leaked due to pressure of carbon dioxide.

### PRIOR ART LITERATURE

### PATENT LITERATURE

Republic of Korea Patent Publication No. 2022-0163559A (Dec. 12, 2022)

### SUMMARY

### TECHNICAL PROBLEM

Embodiments of the present disclosure have been proposed to solve the above problems, and is directed to providing a cosmetic container having a pressure control function capable of controlling pressure of an accommodating space to be lower than a set pressure by discharging gas in the accommodating space to outside in an upright state, a horizontal state, and an inverted state.

In addition, the present disclosure is directed to providing a cosmetic container having a pressure control function that has no risk of rupture or damage even when the pressure of the accommodating space of a body increases.

### TECHNICAL SOLUTION

According to an embodiment, it is possible to provide a cosmetic container having a pressure control function including: a body forming an accommodating space configured to store a content capable of generating gas over time; and a selective blocking member configured to selectively allow the gas to pass to outside according to a magnitude of pressure of the gas generated in the accommodating space.

In addition, it is possible to provide the cosmetic container having a pressure control function, wherein the selective blocking member includes: a cap coupled to a neck portion of the body; an extension part extending from the cap toward the accommodating space and including a first inlet into which the gas generated from the content flows at an extending side end portion; a plug part formed at an end portion of the extension part and including a second inlet communicating with the first inlet; and a valve member formed at the end portion of the extension part and selectively blocking the second inlet to selectively communicate the first inlet and the second inlet.

In addition, it is possible to provide the cosmetic container having a pressure control function, wherein the valve member includes: an elastic member formed across the first inlet; and a protruding member coupled to one side of the elastic member and protruding toward the second inlet of the plug part, wherein the elastic member may be elastically deformed according to the pressure of the gas, and the protruding member selectively blocks the second inlet according to the pressure of the gas.

In addition, it is possible to provide the cosmetic container having a pressure control function, wherein the extension part includes an extension hole extending from the first inlet and connected to the outside.

In addition, it is possible to provide the cosmetic container having a pressure control function further including: an upright state in which the body stands in a gravity direction; a horizontal state in which the body is laid in a direction perpendicular to the gravity direction; and an inverted state in which the body stands in a direction opposite to the upright state, wherein the content is filled in the accommodating space of the body so as not to contact a valve member formed at an end portion of the selective blocking member when the body is in all of the upright state, the horizontal state, and the inverted state.

In addition, it is possible to provide the cosmetic container having a pressure control function, wherein pressure of the accommodating space of the body is maintained lower than a set pressure when the body is in all of the upright state, the horizontal state, and the inverted state.

In addition, it is possible to provide the cosmetic container having a pressure control function, wherein the content includes vitamin C.

In addition, it is possible to provide the cosmetic container having a pressure control function, wherein the extension part is configured to block an opening formed in the neck portion, and an extension hole is formed inside the extension part configured to allow the gas to pass through.

In addition, it is possible to provide the cosmetic container having a pressure control function, wherein the elastic member includes: a first elastic member crossing the first inlet in one direction; and a second elastic member crossing the first inlet in another direction, wherein the protruding member is formed at an intersecting portion of the first elastic member and the second elastic member.

### ADVANTAGEOUS EFFECTS

The cosmetic container having a pressure control function according to one embodiment of the present disclosure is capable of controlling pressure of the accommodating space to be lower than a set pressure by discharging gas in the accommodating space to outside in an upright state, a horizontal state, and an inverted state.

In addition, the cosmetic container having a pressure control function can eliminate a risk of rupture or damage even when the pressure of the accommodating space of the body increases.

### BRIEF DESCRIPTION OF THE DRAWINGS

FIG. 1 is a perspective view of a cosmetic container having a pressure control function according to one embodiment of the present disclosure.

FIG. 2 is a cross-sectional view of the cosmetic container having the pressure control function in FIG. 1.

FIG. 3 is a perspective view of a selective blocking member of the cosmetic container having the pressure control function in FIG. 1.

FIG. 4 is an exploded perspective view of the selective blocking member in FIG. 3.

FIG. 5 is an enlarged view of an end portion of an extension part in FIG. 4.

FIG. 6 is a view conceptually showing an upright state of the cosmetic container having the pressure control function in FIG. 1.

FIG. 7 is a view conceptually showing an inverted state of the cosmetic container having the pressure control function in FIG. 1.

FIG. 8 is a view conceptually showing a horizontal state of the cosmetic container having the pressure control function of FIG. 1.

### DETAILED DESCRIPTION OF THE EMBODIMENTS

Hereinafter, specific embodiments of the present disclosure will be described in detail with reference to the accompanying drawings. In addition, in describing the present disclosure, when it is determined that a detailed description of a related known configuration or function may obscure the gist of the present disclosure, the detailed description will be omitted.

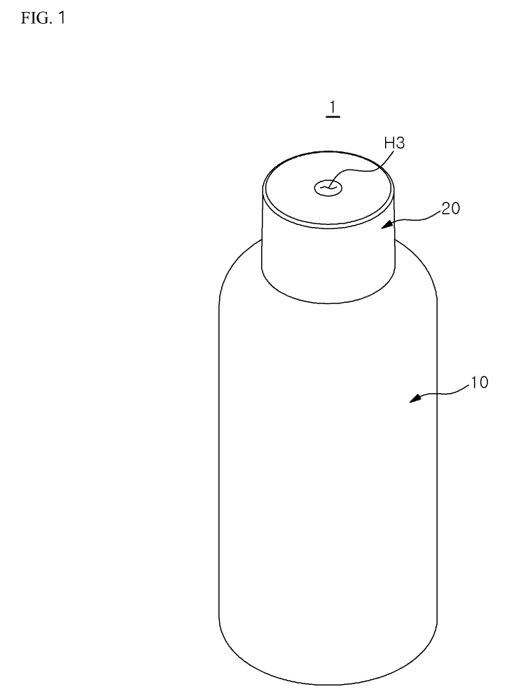

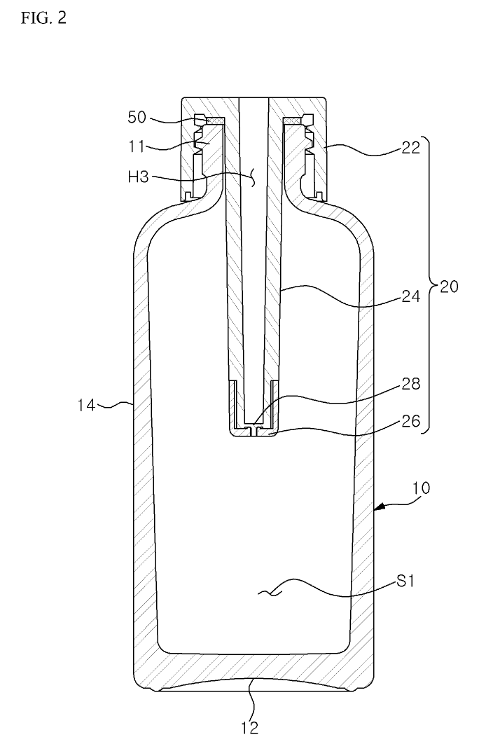

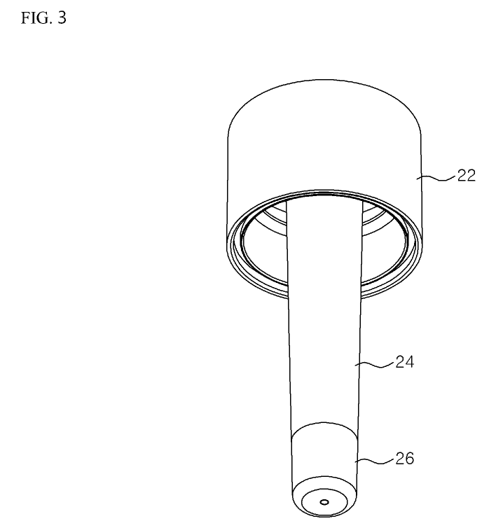

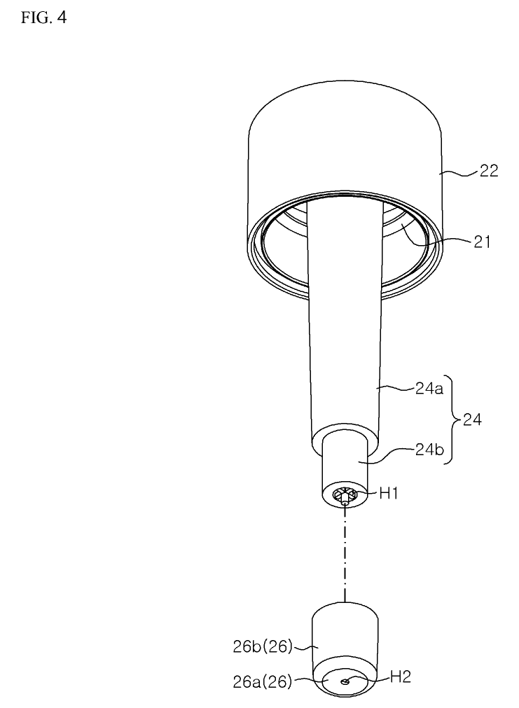

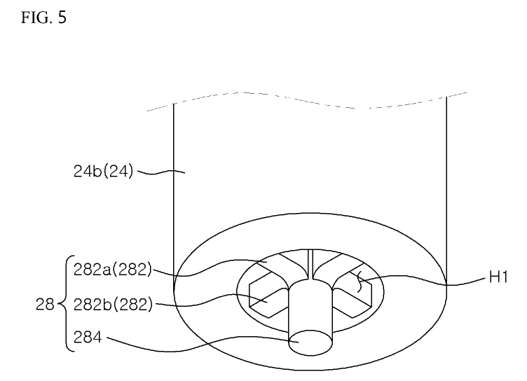

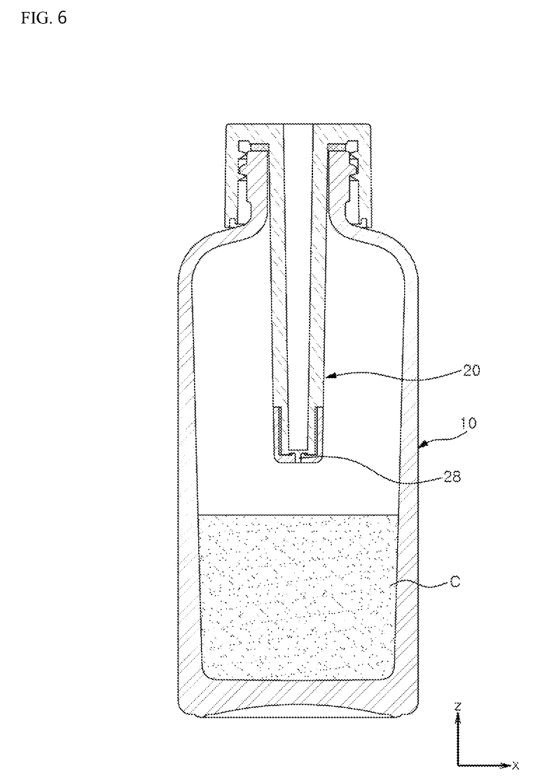

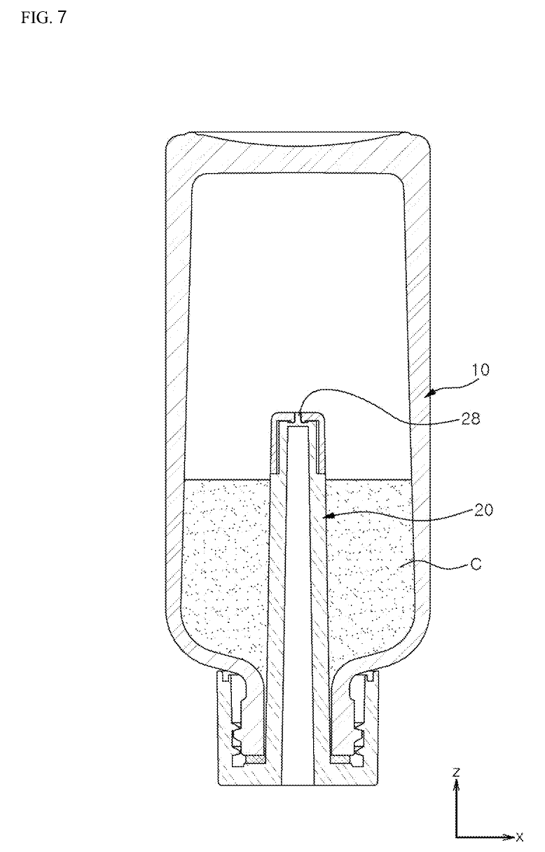

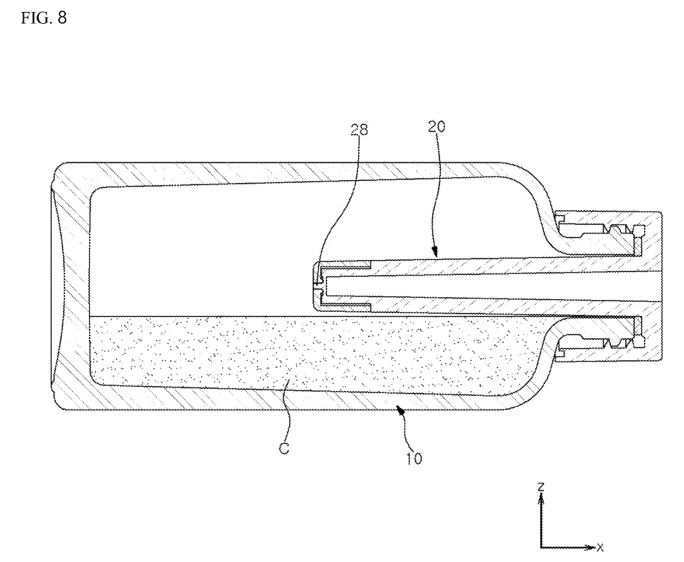

FIG. 1 is a perspective view of a cosmetic container having a pressure control function 1 according to one embodiment of the present disclosure, FIG. 2 is a cross-sectional view of the cosmetic container having the pressure control function 1 in FIGS. 1, FIG. 3 is a perspective view of a selective blocking member 20 of the cosmetic container having the pressure control function 1 in FIGS. 1, FIG. 4 is an exploded perspective view of the selective blocking member 20 in FIGS. 3, FIG. 5 is an enlarged view of an end portion of an extension part 24 in FIG. 4. FIG. 6 is a view conceptually showing an upright state of the cosmetic container having the pressure control function 1 in FIGS. 1, FIG. 7 is a view conceptually showing an inverted state of the cosmetic container having the pressure control function 1 in FIGS. 1, and FIG. 8 is a view conceptually showing a horizontal state of the cosmetic container having the pressure control function 1 of FIG. 1.

Referring to FIGS. 1 - 8, a cosmetic container having a pressure control function 1 according to one embodiment of the present disclosure may include: a body 10 having an accommodating space S1 configured to store a content C capable of generating gas over time; and a selective blocking member 20 configured to selectively allow the gas to pass to outside according to a magnitude of pressure of the gas generated in the accommodating space S1.

The selective blocking member 20 may be formed such that the valve member 28 does not contact the content C when the cosmetic container having the pressure control function 1 is in all of the upright state, the horizontal state, and the inverted state.

By this, the gas generated in the content C may pass through the valve member 28 when the cosmetic container having the pressure control function 1 is in all of the upright state, the horizontal state, and the inverted state and be discharged to the outside of the cosmetic container having the pressure control function 1.

The content C may be filled in the accommodating space S1 so as not to contact the valve member 28 when the cosmetic container having the pressure control function 1 is in all of the upright state, the horizontal state, and the inverted state.

When a user purchases the cosmetic container having the pressure control function 1 for the first time, only a part of the accommodating space S1 of the cosmetic container having the pressure control function 1 may be filled with the content C. For example, the content C may be filled to 50% or less of a volume of the accommodating space S1.

The body 10 may form the accommodating space S1 capable of accommodating the content C, and may include a neck portion 11 that forms an opening through which the content C may be discharged.

The body 10 may be formed in a cylindrical shape. However, the shape of the body 10 is not limited.

The body 10 may include: a bottom surface 12; a side surface 14 extending from the bottom surface 12; and the neck portion 11 extending from the side surface 14 and forming the opening.

The bottom surface 12 may be understood as a surface facing a gravity direction in the upright state, and the side surface 14 may be understood as a surface facing the gravity direction the horizontal state. In the present embodiment, a -z direction may be understood as the gravity direction based on FIG. 6.

In the present embodiment, the upright state may be understood as a state in which the bottom surface 12 of the body 10 contacts an external floor such that the cosmetic container having the pressure control function 1 stands (see FIG. 6), the horizontal state may be understood as a state in which the side surface 14 of the body 10 contacts the external floor such that the cosmetic container having the pressure control function 1 is laid down (see FIG. 8), and the inverted state may be understood as a state in which the selective blocking member 20 contacts the external floor such that the cosmetic container having the pressure control function 1 stands in a direction opposite to the upright state (see FIG. 7).

A thread that may be screw-coupled with a cap 22 of the selective blocking member 20 may be formed on an outer peripheral surface of the neck portion 11, and the opening through which the content C may pass through may be formed inside the neck portion 11.

The body 10 may be formed such that a length in the gravity direction is greater than a length in a direction perpendicular to the gravity direction.

In the present embodiment, the content C may be discharged to the outside through the opening formed at the neck portion 11 of the body 10, and the gas generated in the content C may be discharged to the outside through the selective blocking member 20.

The selective blocking member 20 may be provided to allow only the gas generated in the content C to pass through, and not to allow the content C to pass through. For example, the content C may be filled in the accommodating space S1 so as not to contact the valve member 28 of the selective blocking member 20.

The selective blocking member 20 may include: the cap 22 that is coupled to the neck portion 11 of the body 10; the extension part 24 extending from the cap 22 toward the accommodating space S1 and including a first inlet H1 into which the gas generated from the content C flows at an extending side end portion; a plug part 26 formed at the end portion of the extension part 24 and including a second inlet H2 communicating with the first inlet H1; and a valve member 28 formed at the end portion of the extension part 24 and selectively blocking the second inlet H2 to selectively communicate the first inlet H1 and the second inlet H2.

Here, the cap 22, the extension part 24, and the valve member 28 may be integrally formed, and only the plug part 26 may be formed as a separate member. However, the spirit of the present disclosure is not limited thereto, and the cap 22, the extension part 24, the valve member 28, and the plug part 26 may all be integrally formed.

A thread protrusion 21 that may be coupled to the thread formed at the neck portion 11 may be formed on an inner peripheral surface of the cap 22.

An extension hole H3 extending from the extension part 24 may be formed at a central portion of the cap 22.

The extension part 24 may extend from the cap 22 to the accommodating space S1 of the body 10.

At least a portion of the extension part 24 may be disposed in the accommodating space S1. For example, ⅔ or more of the extension part 24 may be disposed in the accommodating space S1.

A length of the extension part 24 may be provided as ½ or more of a length of the body 10. Here, the length may be understood as a length in a direction in which the extension part 24 extends (z-axis direction).

The extension part 24 may block the opening formed at the neck portion 11.

As the extension part 24 blocks the opening, the content C may be prevented from being discharged to the outside through the opening.

For example, the extension part 24 may be integrally formed with the cap 22, and when a user couples the cap 22 to the neck portion 11, the extension part 24 may block the opening formed at the neck portion 11. Conversely, when the user separates the cap 22 from the neck portion 11, the extension part 24 opens the opening formed at the neck portion 11, and the content C may be discharged to the outside through the opening.

An extension hole H3 configured to allow the gas to pass through may be formed inside the extension part 24, and the extension hole H3 may be connected to the first inlet H1 formed at the end portion of the extension part 24.

The extension part 24 may extend from the first inlet H1 and include the extension hole H3 connected to the outside.

The extension hole H3 may extend starting from the first inlet H1, through the extension part 24, and to the cap 22.

The extension part 24 may be formed to have a step so that the plug part 26 may be coupled thereto.

For example, the extension part 24 may include: a first extension part 24a extending from the cap 22;

and a second extension part 24b extending from the first extension part 24a and of which a cross-section is formed to be smaller than that of the first extension part 24a.

The plug part 26 to be described later may be coupled to the second extension part 24b, and the valve member 28 may be formed at the end portion of the second extension part 24b.

The valve member 28 may be elastically deformed according to the pressure of the gas, and thus the gas may be selectively discharged to the outside of the accommodating space S1. The valve member 28 may be formed at the first inlet H1 of the extension part 24. Here, the first inlet H1 may be understood as a hole formed at the end portion of the extension part 24.

For example, the valve member 28 may include: an elastic member 282 formed by crossing the first inlet H1; and a protruding member 284 coupled to one side of the elastic member 282 and protruding toward the second inlet H2 of the plug part 26.

Here, the elastic member 282 may be elastically deformed according to the pressure of the gas, and the protruding member 284 may selectively block the second inlet H2 according to the pressure of the gas.

When the gas is generated in the content C of the accommodating space S1 and the pressure of the accommodating space S1 reaches a pressure equal to or greater than the set pressure, the gas may push the protruding member 284 blocking the second inlet H2 such that the valve member 28 retreats.

In addition, when the gas is discharged to the outside through the extension hole H3 of the extension part 24 and the pressure of the accommodating space S1 reaches a pressure lower than the set pressure, the valve member 28 moves forward such that the protruding member 284 blocks the second inlet H2.

Here, the retreat may be understood as the valve member 28 moving toward the cap 22, and the forward movement may be understood as the valve member 28 moving toward an opposite side of the cap 22.

The elastic member 282 is disposed at the first inlet H1 formed at the end portion of the extension part 24 and may divide the first inlet H1 into a plurality of holes.

For example, the elastic member 282 may include: a first elastic member 282a crossing the first inlet H1 in one direction; and a second elastic member 282b crossing the first inlet H1 in another direction.

The first elastic member 282a and the second elastic member 282b are formed to overlap each other and may partition the first inlet H1 into four holes.

The elastic member 282 may be disposed at the first inlet H1 and formed as a material that is elastically deformed according to the pressure of the gas. For example, when the pressure of the gas in the accommodating space S1 is equal to or greater than the set pressure, the elastic member 282 may be elastically deformed, and when the pressure of the gas is lower than the set pressure, the elastic member 282 may return to its original state.

When the elastic member 282 is elastically deformed, the protruding member 284 connected to the elastic member 282 may open the second inlet H2, and when the elastic member 282 returns to its original state, the protruding member 284 may seal the second inlet H2.

In addition, the protruding member 284 may be formed at an intersecting portion of the first elastic member 282a and the second elastic member 282b.

The protruding member 284 may be formed to protrude toward the second inlet H2 formed at the plug part 26.

In the present embodiment, it is illustrated that the elastic member 282 is provided as an elastically deformable material and is a member having a shape crossing the first inlet H1, but the spirit of the present disclosure is not limited thereto, and the elastic member 282 may be formed as a spring member.

The valve member 28 may be disposed in the accommodating space S1 of the body 10, but may be disposed at a position that does not contact the content C when the body 10 is in all of the upright state, the horizontal state, and the inverted state.

For example, the valve member 28 may be formed at the second extension part 24b, which is the end portion of the extension part 24, and the second extension part 24b may be disposed at a central portion of the accommodating space S1.

Here, the central portion of the accommodating space S1 may be understood as a position, based on the body 10, that satisfies a center portion of a longitudinal direction (z-axis direction) of the body 10 and a center portion of a side direction (x-axis direction) of the body 10.

The plug part 26 may be coupled to the end portion of the extension part 24 and include the second inlet H2.

Specifically, the plug part 26 may be coupled to the second extension part 24b, which is the end portion of the extension part 24, and block a part of the first inlet H1.

The second inlet H2 may be formed with a smaller diameter than that of the first inlet H1 and be optionally blocked by the protruding member 284.

The plug part 26 may include: a plug part bottom 26a on which the second inlet H2 is formed; and a plug part side surface 26b surrounding the second extension part 24b.

The selective blocking member 20 may form a sealed state in which the accommodating space S1 is blocked from the outside according to internal pressure of the accommodating space S1, and a pressure-relieved state in which the accommodating space S1 is communicated with the outside.

For example, when the accommodating space S1 is lower than the set pressure, the protruding member 284 may block the second inlet H2 to form the sealed state, and when the pressure of the accommodating space S1 is equal to or greater than the set pressure, the protruding member 284 may open the second inlet H2 to form the pressure-relieved state.

Here, the pressure of the accommodating space S1 may vary according to an amount of the gas generated in the content C. For example, as the amount of the gas generated in the content C increases, the pressure of the accommodating space S1 may increase.

In addition, the selective blocking member 20 may selectively seal the opening of the neck portion 11 formed at the body 10 so as to selectively discharge the content C, and may also selectively seal the first inlet H1 formed at the end portion of the selective blocking member 20 so as to selectively discharge the gas.

In addition, an intermediate sealing member 50 may be disposed between the cap 22 of the selective blocking member 20 and the neck portion 11 of the body 10.

The intermediate sealing member 50 may prevent the content C and the gas from leaking between the opening of the neck portion 11 and the cap 22.

The cosmetic container having the pressure control function 1 of the present embodiment may have: the upright state (see FIG. 6) in which the body 10 stands in the gravity direction; the horizontal state (see FIG. 7) in which the body 10 is laid in a direction perpendicular to the gravity direction; and the inverted state in which the body 10 stands in a direction opposite to the upright state.

The selective blocking member 20 may include the valve member 28 that does not contact the content C when the body 10 is in all of the upright state, the horizontal state, and the inverted state.

In this case, a problem of the content C leaking to the outside due to the pressure of the gas may be solved not only when the cosmetic container having the pressure control function 1 is in the upright state, but also when the cosmetic container having the pressure control function 1 is in the horizontal state and the inverted state.

In addition, even when the gas (e.g., carbon dioxide) is generated in the accommodating space S1 of the body 10 and the pressure of the accommodating space S1 increases, the gas may be discharged to the outside through the selective blocking member 20 in all of the upright state, the horizontal state, and the inverted state, thereby preventing the body 10 from rupturing or being damaged.

The content C may be filled in the accommodating space S1 of the body 10 so as not to contact the valve member 28 formed at the end portion of the selective blocking member 20 when the body 10 is in all of the upright state, the horizontal state, and the inverted state.

Here, the content C may include vitamin C, which generates carbon dioxide over time.

In the present embodiment, the content C may be discharged to the outside through the opening formed at the neck portion 11 of the body 10. In addition, the gas may be discharged to the outside through the extension hole H3 formed at the selective blocking member 20. In this case, the protruding member 284 of the valve member 28 and the elastic member 282 coupled thereto may be raised only when the accommodating space S1 is equal to or greater than the set pressure so as to open the second inlet H2.

In the present embodiment, the content C and the gas (e.g., carbon dioxide) generated in the content C may be stored in the accommodating space S1. In this case, the content C may be stored as is in the accommodating space S1, and only the gas may be discharged to the outside of the accommodating space S1, so that the pressure of the accommodating space S1 is maintained lower than the set pressure.

Hereinafter, a process of controlling the pressure of the accommodating space S1 by the cosmetic container having the pressure control function 1 is described.

The content C may be filled so as not to contact the valve member 28 of the selective blocking member 20 in the accommodating space S1. In other words, the content C may be filled so as not to contact the second inlet H2 and the protruding member 284 in the accommodating space S1.

By this, the pressure of the accommodating space S1 may be controlled to be lower than the set pressure when the cosmetic container having the pressure control function 1 is in all of the upright state, the horizontal state, and the inverted state. Here, the set pressure may be controlled differently according to a degree of elasticity of the elastic member 282.

Gas may be generated over time in the content C stored in the accommodating space S1. When the pressure of the accommodating space S1 gradually increases by this and reaches the set pressure, the gas may push the protruding member 284 to open the second inlet H2. By this, the gas stored in the accommodating space S1 may move along the second inlet H2, the first inlet H1, and the extension hole H3 and be discharged to the outside.

In addition, when the gas is discharged to the outside and reaches a pressure lower than the set pressure, the protruding member 284 may block the second inlet H2 by the elasticity of the elastic member 282.

Through this process, the accommodating space S1 of the body 10 may be maintained lower than the set pressure in all of the upright state, the horizontal state, and the inverted state.

Although the cosmetic container having the pressure control function 1 according to the embodiments of the present disclosure has been described as a specific embodiment, this is merely an example, and the present disclosure is not limited thereto, and should be interpreted as having the broadest scope according to the basic spirit disclosed in this specification. Those skilled in the art may combine or substitute the disclosed embodiments to implement embodiments not disclosed herein, but this does not depart from the scope of the present disclosure. In addition, those skilled in the art may easily change or modify the disclosed embodiments based on this specification, and it is clear that such changes or modifications also fall within the scope of the present disclosure.

What is claimed is:

1. A cosmetic container having a pressure control function, comprising:
a body forming an accommodating space configured to store a content capable of generating gas over time; and
a selective blocking member configured to selectively allow the gas to pass to outside according to a magnitude of pressure of the gas generated in the accommodating space.

2. The cosmetic container having a pressure control function of claim 1, wherein the selective blocking member comprises:
a cap coupled to a neck portion of the body;
an extension part extending from the cap toward the accommodating space S1 and including a first inlet H1 into which the gas generated from the content flows at an extending side end portion;
a plug part formed at an end portion of the extension part and including a second inlet communicating with the first inlet; and
a valve member formed at the end portion of the extension part and selectively blocking the second inlet to selectively communicate the first inlet H1 and the second inlet.

3. The cosmetic container having a pressure control function of claim 2, wherein the valve member comprises:
an elastic member formed across the first inlet H1; and
a protruding member coupled to one side of the elastic member and protruding toward the second inlet of the plug part,
wherein the elastic member is elastically deformed according to the pressure of the gas, and the protruding member selectively blocks the second inlet according to the pressure of the gas.

4. The cosmetic container having a pressure control function of claim 2, wherein the extension part comprises an extension hole extending from the first inlet and connected to the outside.

5. The cosmetic container having a pressure control function of claim 1, further comprising:
an upright state in which the body stands in a gravity direction;
a horizontal state in which the body is laid in a direction perpendicular to the gravity direction; and
an inverted state in which the body stands in a direction opposite to the upright state,
wherein the content is filled in the accommodating space of the body so as not to contact a valve member formed at an end portion of the selective blocking member when the body is in all of the upright state, the horizontal state, and the inverted state.

6. The cosmetic container having a pressure control function of claim 5, wherein pressure of the accommodating space of the body is maintained lower than a set pressure when the body is in all of the upright state, the horizontal state, and the inverted state.

7. The cosmetic container having a pressure control function of claim 1, wherein the content includes vitamin C.

8. The cosmetic container having a pressure control function of claim 2, wherein the extension part is configured to block an opening formed in the neck portion, and
an extension hole is formed inside the extension part configured to allow the gas to pass through.

9. The cosmetic container having a pressure control function of claim 3, wherein the elastic member comprises:
a first elastic member crossing the first inlet H1 in one direction; and
a second elastic member crossing the first inlet H1 in another direction,
wherein the protruding member is formed at an intersecting portion of the first elastic member and the second elastic member.
